(19) United States
(12) Patent Application Publication
(10) Pub. No.: US 2011/0286952 A1
(43) Pub. Date: Nov. 24, 2011

(54) DERMOCOSMETIC COMPOSITION, AESTHETIC TREATMENT METHOD USING THE COMPOSITION, AND USE OF THE COMPOSITION FOR LIGHTENING THE PIGMENTATION OF THE SKIN

(76) Inventor: Yvon Gauthier, Le Bouscat (FR)

(21) Appl. No.: 13/055,622

(22) PCT Filed: Jul. 22, 2009
(86) PCT No.: PCT/FR2009/051471

§ 371 Date: Aug. 11, 2011

(30) Foreign Application Priority Data
Jul. 23, 2008 (FR) 0855026

(51) Int. Cl. A61K 8/66; A61Q 19/02; C12N 9/16

(52) U.S. Cl. 424/62; 435/196

(57) ABSTRACT

The invention relates to a dermocosmetic composition essentially characterized in that it comprises at least one hydrolase selected from acid phosphatases.

The invention also relates to a method of aesthetic treatment of the skin aimed in particular at lightening said skin. This method is essentially characterized in that the skin is treated, at least locally, with a composition according to the present invention.

The invention also relates to the use of a composition according to the invention for lightening the skin, in particular for treating pregnancy masks or lentigos.

### PRIORITY CLAIM

The present application is a National Phase entry of PCT Application No. PCT/FR2009/051471, filed Jul. 22, 2009, which claims priority from French Application No. 0855026, filed Jul. 23, 2008, the disclosures of which are hereby incorporated by reference herein in their entirety.

### FIELD OF THE INVENTION

The invention relates, generally speaking, to the field of skin treatment. More precisely, according to one of its aspect, the present invention relates to a dermocosmetic composition aimed in particular at lightening skin pigmentation.

### BACKGROUND OF THE INVENTION

A regular exposition of the skin to the ultraviolet (UV) radiations, as well as other stimuli such as inflammation of mechanical friction, promotes melanin production by melanocytes. The inner mechanism is again badly known. However, it seems that UV stimulates tyrosinase activity, which allows the oxidation of chemical compounds and leads to the synthesis of melanin in melanocytes, which settle on melanosomes. Once melanised, these melanosomes are given to keratinocytes nearby of the melanocytes. These melanosomes bearing melanin remain some time in the keratinocytes cytoplasm.

At the level of the medium and upper layers of the epidermis, in a Caucasian, that is to say in a subject having a white skin, melanised melanomas are then integrated into lysosomes in order to be progressively degraded. This degradation is carried out progressively by the intervention of an enzymatic system contained inside the lysosomes, which includes in particular acid phosphatases, cathepsins, and arylsulfatases.

Usually, in the epidermis of Caucasian subjects, melanin is absent of the stratum corneum because of its progressive destruction in superficial epidermal layers. This is not the case in the epidermis of subjects having a black skin. In these subjects, melanosomes, having a higher size, are not destructed and persist at the level of the stratum corneum.

In some particular cases, a melanotic overload of the epidermal cells and sometimes dermal intracellular melanotic sediments may be observed. This is the case of melasma and lentigos, which are characterised by the apparition of permanent and unsightly hyperpigmented marks.

On the market, there are many preparations having a very variable efficiency for decreasing these hyperpigmentions, which involve a definite aesthetical prejudice.

The objective of these known preparations is, for most of them, to slow down or inhibit melanin production by melanocytes by striking directly at tyrosinase. This is the case of hydroquinone and of many products having a molecular structure close to tyrosine. Some of these products, and for example hydroquinone derivatives, have the disadvantage to be toxic for melanocytes.

Hyperpigmentations may also be decreased by the use of local antioxidants blocking the oxidation of melanin precursors. These antioxidants are essentially ascorbic acid, or sulphurous derivatives.

### BRIEF SUMMARY OF THE INVENTION

In this context, the present invention has as objective to propose an efficient new composition convenient to the lightening of the pigmentation of the skin, and in particular to the lightening of the hyperpigmented marks.

To that purpose, the dermocosmetic composition comprises at least one hydrolase selected from acid phosphatases.

In some aspects, the acid phosphatases are derived from plant origin, such as being from potato, sweet potato, or a cereal plant such as wheat germ.

In some aspects, the dermocosmetic composition may be in the form of a cream, pomade, lotion, gel, milk, pressurised composition for aerosol, solution suitable to be injected, or solution suitable to be administered by ionization.

In some aspects, the dermoscometic composition comprises at least one hydrolase selected from acid phosphatises and at least one lightening agent selected from melanocyte-stimulating hormone inhibitor, endothelin-1 inhibitor, tyrosinase inhibitors, antioxidants, and keratolytic agents.

In some aspects, the dermoscometic composition comprises at least one hydrolase selected from acid phosphatises and at least one tyrosinase inhibitor selected from hydroquinone and its derivatives, kojic acid, arabutin, glabrindin, rucinol, and azelaic acid.

In some aspects, the dermoscometic composition comprises at least one antioxidant selected from ascorbic acid and cysteine and its derivatives.

In some aspects, the dermoscometic composition comprises at least one hydrolase selected from acid phosphatises and at least keratolytic agents selected from glycolic acid and retinoic acid.

The present invention also has the objective of aesthetic treatment of the skin aimed in particular at lightening said skin, the method characterized in that the skin is treated, at least locally, with any of the foregoing dermoscometic compositions.

In some aspects, the method comprises treating the skin by application of the dermoscometic composition onto the epidermis, injection of the dermoscometic composition in the epidermis, or administration of the dermocosmetic composition by ionization.

The dermocosmetic composition of the present invention may be used for lightening the skin, including, treating melasma or lentigos.

### BRIEF DESCRIPTION OF THE DRAWINGS

Further characteristics and advantages of the invention will clearly be brought out from the detail description hereafter, for reference only and without being limitative, in reference to annexed figures in which:

FIGS. 1 and 2 each represents an image seen in optical microscope of a non treated skin sample (FIG. 1, control), or skin sample treated with a composition according to the invention comprising acid phosphatases (FIG. 2);

FIGS. 1 and 2 each represents an image seen in electron microscope of melanomas observed in keratinocytes cytoplasm at the level of a non treated sample (FIG. 3, control), or skin sample treated with a composition according to the invention comprising acid phosphatases (FIG. 4).

### DETAILED DESCRIPTION OF THE INVENTION

The present invention is directed at an efficient new composition convenient to the lightening of the pigmentation of the skin, and in particular to the lightening of the hyperpigmented marks.

The dermocosmetic composition is essentially characterized in that it comprises at least one hydrolase selected from acid phosphatases.

This composition is well tolerated by the skin and allows obtaining a good lightening of the skin within few weeks.

The composition according to the present invention includes in particular between 0.5 and 15% in weight, more preferentially between 5 and 10% in weight, of at least one hydrolase selected from acid phosphatases, cathepsines and arylsulfatases.

Acid phosphatases represent in fact an enzyme family existing both to animal and vegetable kingdoms. In animal kingdom, they are secreted from some cells such as prostate cells, or liver cells. In the vegetable kingdom, they exist in a substantial quantity and their extraction is simpler than from animal tissues.

In a preferred embodiment of the present invention, acid phosphatases are from plant origin. For example, they are extracted from potato, from sweet potato, or from a cereal plant such as wheat germ. The use of acid phosphatases extracted from plant has the advantage to be inexpensive.

Topical use of acid phosphatases seems stimulating the damage of intraepidermal melanin, which results by a lightening of the melasma lesions. In addition, the plant origin acid phosphatase composition according to the invention is particularly well tolerated by the skin.

Cathepsines and arylsulfatases also encompass under this designation several kinds of enzymes.

Cathepsines can be isolated from animal tissues (ox spleen, human placenta, or human liver). In particular, cathepsin D can be used as part of the present invention.

Arylsulfatases, of bacterial origin, can be extracted from Aerobacter aerogenes or Flavobacterium heparinum bacteria, or from animal tissues (escargot (Helix pomatia) or limpet (Patella vulgata)).

The use of acid phosphatases is preferably chosen to the use of Cathepsines and/or arylsulfatases for cost reasons. Indeed, at the present time, acid phosphatases are less expensive than arylsulfatases or Cathepsines. The efficiency of these different families is similar.

According to an advantageous embodiment of the present invention, the dermocosmetic composition can further comprise at least one lightening agent selected from melanocyte-stimulating hormone inhibitor or MSH, endothelin-1 inhibitor or ET-1, tyrosinase inhibitors, antioxidants, and keratolytic agents.

Preferably, such a composition according to the present invention comprises between 0.5 and 15% in weight, more preferentially between 5 and 10% in weight, of at least one hydrolase selected from acid phosphatases, cathepsines and arylsulfatases, and between 0.5 and 5% in weight of at least one lightening agent selected from melanocyte-stimulating hormone inhibitor or MSH, endothelin-1 inhibitor or ET-1, tyrosinase inhibitors, antioxidants, and keratolytic agents.

In particular, the composition can comprise, as lightening agent, at least a tyrosinase inhibitor selected from hydroquinone and its derivatives, kojic acid, arabutin, glabrindin, rucinol, and azelaic acid, and/or comprise at least an antioxidant selected from ascorbic acid and cysteine and its derivatives, and/or comprise a keratolytic agent selected from glycolic acid and retinoic acid.

The combination in a same dermocosmetic composition of a product stimulating melanin degradation, such as acid phosphatases, cathepsines, or arylsulfatases, and a product slowing down melanin synthesis, such as known lightening agents, shows an improved efficiency and tolerance in comparison with those containing only such known lightening agents.

The composition according to the present invention can be in one of the following forms: cream, pomade, lotion, gel, milk, pressurised composition for aerosol, solution suitable to be injected, or solution suitable to be administered by ionization.

The composition according to the present invention includes, in addition to the acid phosphatase and possible at least one lightening agent, one or more additives commonly used in dermocosmetic compositions.

According to a second aspect, the present invention relates to a method of aesthetic treatment of the skin aimed in particular at lightening said skin, characterized in that the skin is treated, at least locally, with a composition according to the present invention.

According to an embodiment of the present invention, the skin is treated by application of said composition onto the epidermis.

According to another embodiment of the present invention, the skin is treated by injection of said composition in the epidermis.

According to a further embodiment of the present invention, the skin is treated by administration of said composition by ionization.

According to a third aspect, the present invention relates to the use of a composition according to the present invention for lightening the skin, and for example for treating melasma or pregnancy masks, or lentigos (senile lentigos or solar lentigos).

The dermocosmetic composition according to the invention can also be used for lightening black skins.

### EXAMPLES

### Example 1

### Use of Acid Phosphatases From Plant Origin For Lightening the Skin

### Example 1A

### Studies In Animal

Experimental studies have been carried out on Guinea pigs having a black hair coat.

Twice daily local applications of solution comprising 5% in weight of acid phosphatases have been applied onto the right ear of the Guinea pigs, the left ear being the control.

These solutions also contained water and an excipient allowing the penetration of the solution in epidermis, in the present example, propylene glycol.

A lightening notable to the naked eye has been observed within 15 days.

In addition, comparative histological studies, in optical microscope, have been carried out on a sample (2 mm diameter punch) of treated ears and non treated ears.

Cutaneous samples of treated ears and non treated ears have also been used to achievement of ultrastructural study in electron microscopy.

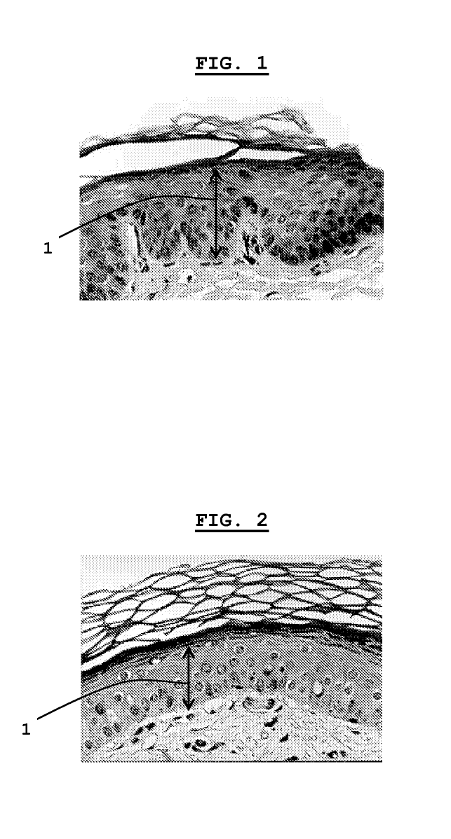

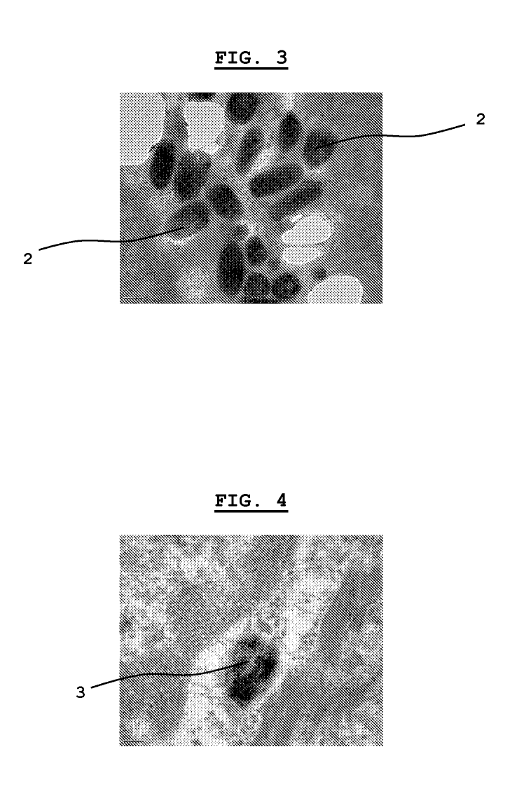

FIG. 2 shows a disappearance of dark areas at the level of epidermis 1 in comparison with FIG. 1. In other words, a significant decrease of the melanin in epidermis 1 has been observed on treated areas, said decrease that can be related to the lightening of the skin observed at naked eye.

FIG. 3 shows a non treated sample in which melanomas 2 have a homogeneous structure, whereas after treatment, FIG. 4 reveals a melanoma 3 having a heterogeneous structure instancing an ongoing digestion process of the melanin.

These studies have confirmed that disappearance of epidermal melanin coexisting with a normal synthesis of melanin by melanocytes in the epidermis at the level of treated areas. This tends to prove that local provision of acid phosphatase has favoured melanin degradation.

In animals, good efficiency of the lightening and good tolerance of the composition have been obtained.

### Example 1B

### Studies In Human

A partial study has been carried out on subject having melasma and not having responded to current treatments existing in the market.

A solution of acid phosphates identical to the one of example 1A has been applied onto facial areas to be treated once a day.

It has been bringing out that the tolerance of the acid phosphatase solution was perfect and that lightening effect was good. For example, alleviation of hyperpigmentation of melasma has been observed within three weeks in 70% of the cases.

### Example 2

### Use of A Mixture of Acid Phosphatase And A Lightening Agent For Lightening the Skin

A solution containing a mixture of acid phosphatase and N-acetylcysteine as lightening agent has been used in studies in animal and human shown in examples 2A and 2B.

The solution contained 5% in weight of acid phosphatases of plant origin and 5% in weight of N-acetylcysteine, in water and propylene glycol.

### Example 2A

### Studies In Animal

The same protocol as in example 1A has been followed with a solution containing a mixture of acid phosphatase and N-acetylcysteine instead of a solution of acid phosphatases without additional lightening agent.

In animals, the efficiency of lightening of a mixture of acid phosphatases extracted from plants and of N-acetylcysteine is more important that the efficiency of the isolated solution of acid phosphatases.

On the opposite, tolerance is not quite as good because of the presence of a cysteine derivative, which is often irritating for the skin.

### Example 2B

### Studies In Human

The same protocol as in example 1B has been followed with a solution containing a mixture of acid phosphatase and N-acetylcysteine instead of a solution of acid phosphatases without additional lightening agent, in subject having resistant melanomas.

Results obtained in animal (example 2A) have thus been confirmed in human.

With such a composition, which at the same time promotes melanin degradation and slows down its synthesis, the lightening efficiency is increased, but tolerance is not as good as the tolerance observed with a composition containing only acid phosphatases without additional lightening agent.

1. A dermocosmetic composition comprising at least one hydrolase selected from acid phosphatases.

2. The dermocosmetic composition according to claim 1, wherein acid phosphatases are from plant origin.

3. The dermocosmetic composition according to claim 1, wherein acid phosphatases are extracted from a potato, a sweet potato, or a wheat germ cereal plant.

4. The dermocosmetic composition according to claim 1 wherein the composition is in the form of a cream, pomade, lotion, gel, milk, pressurised composition for aerosol, solution suitable to be injected, or solution suitable to be administered by ionization.

5. The dermocosmetic composition according to claim 1, further comprising at least one lightening agent selected from melanocyte-stimulating hormone inhibitor, endothelin-1 inhibitor, tyrosinase inhibitors, antioxidants, and keratolytic agents.

6. The dermocosmetic composition according to claim 5, wherein the at least one lightening agent comprises at least one tyrosinase inhibitor selected from hydroquinone and its derivatives, kojic acid, arabutin, glabrindin, rucinol, and azelaic acid.

7. The dermocosmetic composition according to claim 5, wherein the at least one lightening agent comprises at least one antioxidant selected from ascorbic acid and cysteine and its derivatives.

8. The dermocosmetic composition according to claim 5, a wherein the at least one lightening agent comprises at least one keratolytic agent selected from glycolic acid and retinoic acid.

9. A method of aesthetic treatment of the skin aimed at lightening said skin, the method comprising:
treating the skin, with a composition comprising at least one hydrolase selected from acid phosphatases.

10. The method according to claim 9, wherein the skin is treated by application of said composition onto the epidermis.

11. The method according to claim 9, wherein the skin is treated by injection of said composition in the epidermis.

12. The method according to claim 9, wherein the skin is treated by administration of said composition by ionization.

13. A method for treating melasma, or lentigos, the method comprising:
applying to a subject in need thereof a composition comprising at least one hydrolase selected from acid phosphatases.

14. (canceled)
